(19) United States
(12) Patent Application Publication
(10) Pub. No.: US 2019/0365347 A1
(43) Pub. Date: Dec. 5, 2019

(54) ULTRASONIC PROBE AND ULTRASONIC IMAGE OBTAINING APPARATUS

(71) Applicant: CANON KABUSHIKI KAISHA, Tokyo (JP)

(72) Inventor: Naoto Abe, Machida-shi (JP)

(21) Appl. No.: 16/363,834

(22) PCT Filed: Nov. 30, 2017
(86) PCT No.: PCT/JP2017/043007

§ 371 Date: Mar. 25, 2019

(30) Foreign Application Priority Data
Dec. 8, 2016 (JP) 2016-238662

(51) Int. Cl. A61B 8/14; A61B 8/00; G01N 29/06; G01N 29/24

(52) U.S. Cl. CPC A61B 8/14; G01N 29/2418; G01N 29/0672; A61B 8/4444

(57) ABSTRACT

A hand-held type ultrasonic probe includes a housing having a reception region portion and a grip portion. The reception region portion includes an ultrasonic element array which receives ultrasonic waves and the grip portion includes a vibration actuator.

### CROSS-REFERENCE TO RELATED APPLICATIONS

This application is a Continuation of International Patent Application No. PCT/JP2017/043007, filed Nov. 30, 2017, which claims the benefit of Japanese Patent Application No. 2016-238662, filed Dec. 8, 2016, both of which are hereby incorporated by reference herein in their entirety.

### TECHNICAL FIELD

The present invention relates to an ultrasonic probe and an ultrasonic image obtaining apparatus.

### BACKGROUND ART

In recent years, photoacoustic imaging apparatuses which generate an image of an inside of a living body utilizing a photoacoustic effect have been studied and developed. Such a photoacoustic imaging apparatus generates an image of an inside of a test object using an ultrasonic wave (a photoacoustic wave) generated from a tissue of the living body which absorbs energy of pulse laser light emitted to the inside of the living body due to the photoacoustic effect. On the other hand, ultrasonic imaging apparatuses (ultrasonic image obtaining apparatuses) which have a probe transmitting an ultrasonic wave and which generates an image based on the ultrasonic wave reflected by an inside of a living body have been used. These apparatuses have a common technique of receiving an ultrasonic wave from an inside of a living body and generating an image of the inside of the living body, and therefore, an imaging apparatus which has functions of the two types of imaging apparatus have also been studied and developed. Furthermore, an ultrasonic imaging apparatus which includes a probe which may be held by a hand which is referred to as a hand-held type ultrasonic probe is dominant. PTL 1 discloses an imaging apparatus having the functions of the two types of imaging apparatus described above using a hand-held type ultrasonic probe.

### CITATION LIST

### Patent Literature

PTL 1 Japanese Patent Laid-Open No. 2012-152267

PTL 2 Japanese Patent Laid-Open No. 2004-16268

PTL 3 Japanese Patent Laid-Open No. 2014-61137

In PTL 1, a configuration in which a hand-held type ultrasonic probe has a switch for selecting photoacoustic imaging or ultrasonic imaging is disclosed. On the other hand, when a photoacoustic image or an ultrasonic image is obtained using a hand-held type ultrasonic probe, the probe is required to be in contact with a test object, e.g., a patient. The inventor discusses improvement of usability of the hand-held type ultrasonic probe, and concluded that, when measurement is performed by bringing the probe into contact with the patient, it is effective to give some information except for visual information to an operator in accordance with a state of the measurement, a state of a probe (in a danger case), or the like. Then the inventor thought that a mechanism for transmitting information to the operator by a tactile impression of the ultrasonic probe contributes to improvement of a technique of this field. Accordingly, an object of the present invention is to provide a hand-held type ultrasonic probe capable of transmitting information to an operator by a tactile impression.

### SUMMARY OF INVENTION

A hand-held type ultrasonic probe according to the present invention includes a housing having a reception region portion and a grip portion. The reception region portion includes an ultrasonic element array which receives ultrasonic waves and the grip portion includes a vibration actuator.

Further features of the present invention will become apparent from the following description of exemplary embodiments with reference to the attached drawings.

### BRIEF DESCRIPTION OF DRAWINGS

FIG. 1 is a diagram illustrating a configuration of an example of a hand-held type ultrasonic probe according to the present invention.

FIG. 2 is a diagram illustrating a configuration of an example of another hand-held type ultrasonic probe according to the present invention.

FIG. 3 is a diagram illustrating a configuration of an example of a further hand-held type ultrasonic probe according to the present invention.

FIG. 4 is an entire block diagram illustrating an example of an ultrasonic image obtaining apparatus including the probe according to the present invention.

FIG. 5 is a timing chart of an image obtainment when the ultrasonic image obtaining apparatus according to the present invention is employed.

FIG. 6 is an entire block diagram illustrating an example of the other ultrasonic image obtaining apparatus.

FIG. 7A is a diagram schematically illustrating the hand-held type ultrasonic probe according to the present invention which is moved in an inclined state.

FIG. 7B is a diagram schematically illustrating the hand-held type ultrasonic probe according to the present invention which is moved in the inclined state.

FIG. 8 is a diagram schematically illustrating an example of display performed by a display unit of the ultrasonic image obtaining apparatus according to the present invention.

FIG. 9A is a diagram schematically illustrating a change of a display state of a message area.

FIG. 9B is a diagram schematically illustrating the change of the display state of the message area.

### DESCRIPTION OF EMBODIMENTS

Hereinafter, preferred embodiments of the present invention will be described with reference to the accompanying drawings. Note that sizes, quality of materials, shapes, and relative arrangement of components described below are to be appropriately changed depending on a configuration of an apparatus to which the present invention is applied and various conditions. Therefore, the scope of the present invention is not limited to the description below.

The present invention relates to a technique of detecting an acoustic wave transmitted from a test object and generating and obtaining characteristic information of an inside of the test object.

The present invention includes an apparatus (a photoacoustic apparatus) which receives an acoustic wave generated in an inside of a test object by irradiating the test object with light and obtaining characteristic information of the test object as image data utilizing a photoacoustic effect. The characteristic information of the present invention indicates information on characteristic values corresponding to different positions in the test object which are generated using photoacoustic signals obtained when ultrasonic waves are received.

The characteristic information obtained in the present invention indicates values which reflect absorption rates of optical energy. For example, a generation source of an acoustic wave generated due to light emission, an initial sound pressure in the test object, and optical energy absorption density and an optical energy absorption coefficient which are derived from the initial sound pressure may correspond to as “characteristic information based on optical absorption” or “distribution of optical characteristic values in an inside of a test object”. Examples of the characteristic information further include concentration associated information of subjects included in a tissue.

The concentration associated information includes a value associated with concentration of a substance incorporated in the test object which is obtained using the characteristic information based on optical absorption for a plurality of wavelengths. Specifically, the concentration associated information includes a degree of oxygen saturation, a value obtained by weighting intensity of an absorption coefficient or the like to the degree of oxygen saturation, total hemoglobin concentration, oxyhemoglobin concentration, and deoxyhemoglobin concentration. Furthermore, the concentration associated information may be glucose concentration, collagen concentration, melanin concentration, or volume fraction of fat or water. Furthermore, a 2D or 3D characteristic information distribution is obtained based on the concentration associated information in the different positions in the test object. Distribution data may be generated as image data.

A main object of the photoacoustic imaging apparatus (a photoacoustic image obtaining apparatus) in the embodiments below is to make diagnosis of a malignant tumor and a vascular disease of a person and an animal, to perform follow-up of chemical treatment, and the like. Accordingly, it is assumed that the test object is a portion of a living body, that is, a portion (a breast, an organ, a circulatory organ, a digestive organ, a bone, a muscle, fat, or the like) of a person or an animal. Examples of a substance of an inspection target include hemoglobin, glucose, water in a body, melanin, collagen, and fat. Furthermore, any substance including a contrast agent, such as indocyanine green (ICG), which is internally administered, may be employed as long as an optical absorption spectrum of the substance is characteristic.

The present invention is applicable not only a photoacoustic imaging apparatus but also various apparatuses including a hand-held type probe for receiving an ultrasonic wave. Furthermore, the present invention includes an imaging apparatus (an image obtaining apparatus) including a hand-held type ultrasonic probe having a vibration actuator inside thereof. The present invention further includes a method for transmitting a message to an operator using an imaging apparatus. The method includes a step of vibrating a vibration actuator in a vibration pattern corresponding to content of the message.

### First Embodiment

Apparatus Configuration

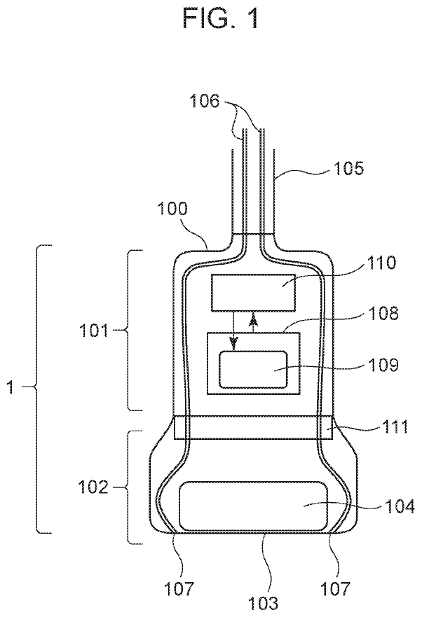

FIG. 1 is a diagram illustrating a configuration of an example of a hand-held type ultrasonic probe according to a first embodiment of the present invention. In FIG. 1, a hand-held type ultrasonic probe 1 includes a housing 100 of the hand-held type ultrasonic probe 1, a grip portion 101 of the hand-held type ultrasonic probe 1 which is held by an operator, and a reception region portion 102 having a probe which receives an ultrasonic wave transmitted from a test object described below. The hand-held type ultrasonic probe 1 further includes a contact surface 103 which is to be brought into contact with the test object and probe elements (an ultrasonic element array) 104 which receive ultrasonic waves transmitted from the test object (photoacoustic waves generated in the test object due to light irradiation) and which are disposed in the reception region portion 102. As the probe elements 104, an ultrasonic transducer including an element which performs mechanoelectric conversion based on an ultrasonic wave may be used. As a concrete example, a probe using a capacitive micromachined ultrasonic transducer: CMUT) element or a piezoelectric element may be employed. Furthermore, the probe elements 104 are disposed in a single line as 1D arrangement or in a plurality of lines as 2D arrangement or on a frame of a spherical surface so as to have sensitivity on inner sides thereof, and are implemented such that the probe elements 104 have the sensitivity directed to an outside of the probe 1. Furthermore, in a case where a photoacoustic imaging apparatus and an ultrasonic imaging apparatus which are described below are switched from one to another, transmission and reception of an ultrasonic wave may be performed using the same probe element, or different probe elements which are optimized for different functions may be disposed.

A cable 105 is connected to a body of the imaging apparatus and optical fibers 106 are bundle fibers, for example, which guide light pulses transmitted from a light source of the imaging apparatus. The cable 105 is configured by integrating the optical fibers 106, analog outputs (ultrasonic reception signals) of the probe elements 104, and lines which transmit information between a controller 201 and a microcomputer 110 as a single line. Since the body of the imaging apparatus and the hand-held type ultrasonic probe 1 may be connected to each other only by the single line, operability of the probe 1 is improved. A light irradiation unit (a light emission terminal) 107 which irradiates the test object with guided laser light is disposed on the contact surface 103 in a tip end portion of the cable 105 includes a diffuser panel and a lens optical system.

A pressure sensor 108 serves as a switch, and a vibration actuator 109 is one of characteristic points of the present invention and is disposed on a back surface of the pressure sensor 108 or in the vicinity of the pressure sensor 108. The microcomputer 110 realizes a function of a switch similar to that described in PTL 1 by determining an output of the pressure sensor 108 and controlling an operation of the vibration actuator 109. Furthermore, the microcomputer 110 transmits an output which is a switch signal to the body of the imaging apparatus through the cable 105. The functions of the microcomputer 110 may be performed by a controller of the body of the imaging apparatus described below. In this case, although the number of electric lines of the cable 105 is increased, the microcomputer 110 is not required to be implemented in the grip portion 101 of the hand-held type ultrasonic probe. A vibration-free member 111 is included in a vibration-free mechanism and suppresses transmission of vibration of the vibration actuator 109 to the reception region portion 102. An anti-vibration rubber, an anti-vibration gel, or the like is suitably used for the vibration-free member 111. The vibration-free member 111 suppresses transmission of vibration by converting the vibration into thermal energy. The vibration-free member 111 is useful in a case where noise is generated when vibration of the vibration actuator 109 is added to an output of the probe element in addition to an ultrasonic wave which is to be received when the vibration actuator 109 is implemented in the hand-held type ultrasonic probe 1 which receives ultrasonic waves.

Here, a function of generating an image of functional information by receiving a photoacoustic wave generated by light irradiation and a function of generating an image of structural information based on a reflection rate of an ultrasonic wave using the ultrasonic wave which has been transmitted, reflected, and returned may be switched from one to another in response to an instruction for operating the pressure sensor 108 disposed on the hand-held type ultrasonic probe 1.

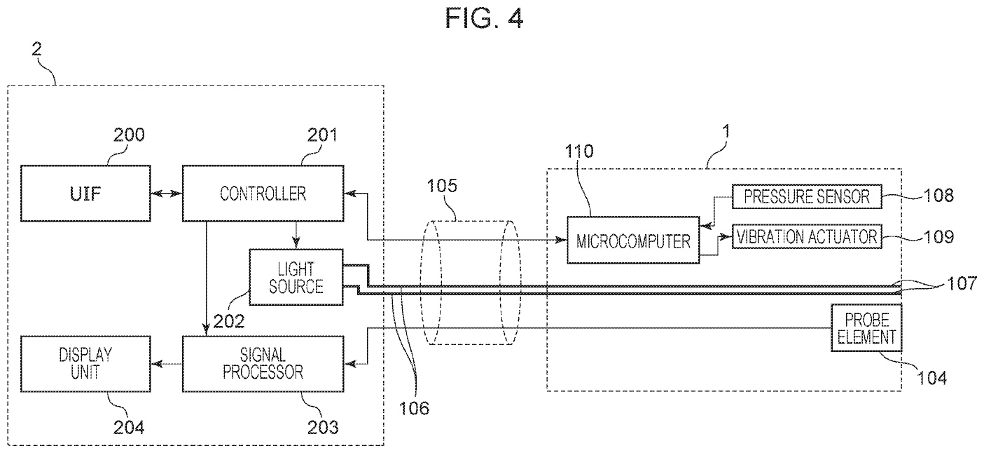

FIG. 4 is a block diagram of the entire photoacoustic image obtaining apparatus (the photoacoustic imaging apparatus) according to the first embodiment of the present invention. In FIG. 4, descriptions of portions which have reference numerals the same as those described above are omitted. In FIG. 4, the body of the image obtaining apparatus (a body of the imaging apparatus) 2 according to the first embodiment of the present invention is connected to the hand-held type ultrasonic probe 1 through the cable 105. Although not illustrated, the cable 105 may include a connector so that the hand-held type ultrasonic probe 1 and the imaging apparatus body 2 may be separated from each other. Specifically, the ultrasonic probe 1 connected to the cable 105 through the connector may be connected to the apparatus body 2 and the ultrasonic probe 1 may be separated from the apparatus body 2. Alternatively, the imaging apparatus body 2 may include a plurality of connectors so that a plurality of hand-held type ultrasonic probes are implemented and one of the probes may be selected where appropriate. Specifically, a plurality of ultrasonic probes having different characteristics may be connected to different cables and the plurality of ultrasonic probes may be connected to the apparatus body 2 with the cables in a replacement available manner.

The user interface 200 is used to input an instruction issued by the operator using a mouse, a keyboard, a touch panel or the like. The controller 201 controls the entire system based on an instruction issued by the operator using the user interface 200. The controller 201 will be described in detail hereinafter. A light source 202 emits a light pulse, and a solid-state laser, such as a titanium sapphire laser, a semiconductor laser, or a light emitting diode (LED) may be employed, for example. A signal processor 203 amplifies analog outputs (ultrasonic reception signals) of the probe elements 104 and converts the analog outputs into digital signals using an analog-digital converter (ADC). Then the signal processor 203 calculates ultrasonic image data and photoacoustic image data using the digital signal obtained by the analog-digital conversion. Specifically, the signal processor 203 performs a process of generating image data in accordance with a signal based on a received ultrasonic wave. A personal computer (PC) or a work station which includes calculation resources, such as a central processing unit (CPU), a graphic processing unit (GPU), and a storage device, or dedicated hardware using a field-programmable gate array (FPGA) or the like is suitable for the signal processor 203.

To obtain an ultrasonic image, after ultrasonic waves are transmitted from the probe elements 104, based on the digital signals obtained by converting the analog outputs (the ultrasonic reception signals) of the probe elements 104 using the ADC, B mode image data for calculating a distance and brightness using a period of time (a reflection time) between a time when the ultrasonic waves are transmitted from the probe elements 104 and a time when the ultrasonic reception signals are received and intensity of the received ultrasonic reception signals is generated or image data is generated by a pseudo color process (a Doppler method) of converting a frequency change of a ultrasonic wave in a certain point into a flow rate. On the other hand, to obtain a photoacoustic image, the signal processor 203 performs a reconfiguration process based on the digital signals of the plurality of probe elements 104 which have received after the optical pulse emission so as to generate reconfiguration image data (characteristic information) of the inside of the test object. As the reconfiguration process, an arbitrary general method, such as Delay and Sum, a back projection method, or a Fourier transform method may be used. Furthermore, reconfiguration image data of concentration associated information of a substance included in a tissue may be calculated using light pulses of different wavelengths. A display unit 204 displays ultrasonic image data and photoacoustic image data generated by the signal processor 203 and is realized by a liquid crystal monitor or an electroluminescence (EL) monitor, for example. Note that the generated image data may be output to a network or simply stored in a nonvolatile memory or the like.

Operation of Apparatus

An operation of the imaging apparatus which performs control of a switching operation using the pressure sensor 108 of the hand-held type ultrasonic probe 1 according to the first embodiment will now be described. The hand-held type ultrasonic probe 1 described in this example may perform switching between a photoacoustic (PA) mode for obtaining a photoacoustic image and an ultrasonic (US) mode for obtaining an ultrasonic image by transmitting an instruction to the pressure sensor 108.

The microcomputer 110 detects a pressure obtained by the pressure sensor 108 and operates as below. In a case where a small pressure is applied to the pressure sensor 108 (a soft touch), the microcomputer 110 notifies the operator of a current switch state by vibration of the vibration actuator 109. In the US mode, for example, vibration occurs for a short period of time. On the other hand, in the PA mode, the vibration actuator 109 notifies the operator of the current mode by vibration for a long period of time. The switching operation is realized when the pressure sensor 108 is pressed hard. The microcomputer 110 detects a pressure applied to the pressure sensor 108. When a large pressure is applied (a hard press), the current mode is changed. That is, in a case where the pressure sensor 108 is pressed hard in the PA mode, a switch state is changed to the US mode. On the other hand, in a case where the pressure sensor 108 is pressed hard in the US mode, a switch state is changed to the PA mode. Here, when the pressure sensor 108 is pressed, the vibration actuator 109 is activated such that the operator feels a clicking sensation, and thereafter, vibration lasts for a short period of time in the US mode or vibration lasts for a longer period of time in the PA mode. The microcomputer 110 performs such operation control so as to output the switch state to the imaging apparatus body 2 through the cable 105. Furthermore, an operator or a patient is put in danger, if the hand-held type ultrasonic probe 1 is separated from an affected area while the light source 202 outputs a light pulse in the PA mode, and therefore, the light pulse which is leaked from a gap is incident on an eye of the operator or the patient. Accordingly, it is preferable that the microcomputer 110 controls the vibration actuator 109 so that the vibration actuator 109 continuously or intermittently generates imperceptible vibration as an alert.

Furthermore, an imaging apparatus having a PA/US mode for repeatedly performing an obtainment of a photoacoustic image and an obtainment of an ultrasonic image in addition to the PA mode and the US mode may be realized. In this case, the microcomputer 110 detects a pressure applied to the pressure sensor 108 and operates as below. In a case where a small pressure is applied to the pressure sensor 108 (a soft touch), the microcomputer 110 notifies the operator of a current switch state by vibration of the vibration actuator 109. In addition to the vibration patterns of the modes described above, in the PA/US mode, the vibration actuator 109 notifies the operator of the current mode by performing vibration for a short period of time, a short break, and vibration for a long period of time. The switching operation is realized when the pressure sensor 108 is pressed hard. The microcomputer 110 detects a pressure applied to the pressure sensor 108. When a large pressure is applied (a hard press), the current mode is successively changed from the PA mode, the US mode, the PA/US mode, the PA mode, and so on. Here, when the pressure sensor 108 is pressed, the vibration actuator 109 is operated such that the operator feels a clicking sensation, and thereafter, vibration lasts for a short period of time in the US mode, vibration lasts for a longer period of time in the PA mode, and vibration for a short period of time, a short break, and vibration for a long period of time are performed in the PA/US mode. For example, in a case where the first and second modes are simultaneously operated, vibration may be performed in a third vibration pattern which is different from a first vibration pattern in which the vibration actuator 109 is vibrated during the first mode and a second vibration pattern in which the vibration actuator 109 is vibrated during the second mode.

The microcomputer 110 performs such operation control so as to output the switch state to the imaging apparatus body 2 through the cable 105. The vibration patterns of the vibration actuator 109 described above are merely examples and other vibration patterns may be employed as long as the individual vibration patterns are unique.

In the configuration described above, the single pressure sensor 108 and the single vibration actuator 109 are implemented in the hand-held type ultrasonic probe 1. According to the present invention, different functions may be realized in different portions in a sensitive surface of the pressure sensor 108 by providing a large sensitive area of the pressure sensor 108 and disposing a plurality of pressure sensors 108 and a plurality of vibration actuators 109, for example. For example, in addition to the mode switching function described above, a function of performing control of turning on a laser in the PA mode may be realized. The operator may discriminate different functions by feeling different vibrations of the vibration actuators 109 disposed in the different portions. Also in this case, the discrimination may be performed using different vibration patterns. According to the present invention, even if the vibration-free member (vibration-free mechanism) 111 is not disposed, noise of an output of the probe element 104 or an uncomfortable feeling of the patient may be suppressed by devising a vibration direction of the vibration actuator 109, an attachment position of the grip portion 101, and vibration patterns. For example, the housing 100 is generated using a material of high attenuation rate relative to vibration. As another method, amplitude of vibration is minimized so that an uncomfortable feeling of the patient is removed, and only frequency components except for frequency components required for generating an ultrasonic image and a photoacoustic image are used as a vibration pattern. Specifically, if the vibration actuator 109 does not generate vibration in frequencies equal to or larger than 1 kHz, noise of an ultrasonic image and a photoacoustic image may be separated in a frequency domain. That is, this is easily realized using an analog/digital high-pass filter.

In other words, vibration is controlled so that a vibration spectrum generated by the vibration actuator 109 is obtained in a frequency band other than a frequency band of a signal (a signal in a reception band) which is transmitted from the probe element 104 and which has a frequency component required for processes performed on the ultrasonic image and the photoacoustic image. Specifically, control may be performed such that a frequency band used by an ultrasonic element array included in the probe element 104 for reception of an ultrasonic wave and a frequency band for vibration of the vibration actuator 109 are different from each other. Furthermore, when it is assumed that a frequency band of the probe element 104 has a frequency characteristic corresponding to a received ultrasonic wave and a portion of maximum receiving sensitivity is 100, the vibration actuator 109 preferably vibrates in a spectrum equal to or lower than a frequency corresponding to 10% (preferably 3%) of the maximum receiving sensitivity or less. Specifically, when it is assumed that maximum sensitivity in a frequency band used for reception of an ultrasonic wave by the ultrasonic element array is set to 100, it is preferable that the vibration actuator 109 is vibrated in a frequency band corresponding to 10% of the maximum sensitivity or less.

To remove frequency components in which vibration is not required in the vibration actuator 109 in which a movement moves in a reciprocal manner, a method for implementing a damper or the like in a stop portion of the movement of the vibration actuator 109 so that vibration in a high frequency caused by collision of mechanism members is suppressed. Furthermore, the frequency components in which vibration is not required may be removed by driving the movement in a driving waveform which does not include high frequency components (driving the movement in a driving waveform which is similar to a sine wave instead of driving in a rectangle wave, for example). Furthermore, the vibration actuator 109 may be disposed so that vibration applied to the probe element 104 and the patient is reduced taking orientation of generation of the vibration of the vibration actuator 109 into consideration. This method is also effective and inexpensive. As another method, a method for implementing portions of the vibration actuator 109 other than portions touched by the operator in the housing 100 in a state in which the portions are surrounded by a vibration-free member may also suppress generation of noise in an output of the probe element 104 and an uncomfortable feeling of the patient. Furthermore, as for noise which may not be removed, an ultrasonic reception signal may be subjected to an analog filter process so that noise is effectively removed in a frequency domain or a digital signal which has been converted by the ADC may be subjected to a digital filtering process so that noise is effectively removed in the frequency domain. Furthermore, a noise generation time point of the vibration actuator 109 is managed by the microcomputer 110, and therefore, the signal processor 203 may record only a signal waveform of noise and may subtract the waveform of the noise from the ultrasonic reception signal by an analog process or a digital process (a process in a time domain).

According to the first embodiment of the present invention, since the vibration actuator 109 is disposed on the grip portion 101 and the vibration-free member 111 is implemented between the vibration actuator 109 and the reception region portion 102, transmission of vibration of the vibration actuator 109 to the patient through the probe element 104 and the contact surface 103 is suppressed. Consequently, generation of noise in an output of the probe element 104 and an uncomfortable feeling of the patient may be suppressed.

### Second Embodiment

A second embodiment of the present invention made by improving the first embodiment is effective in a case where generation of noise in an output of a probe element 104 may not be suppressed or a case where suppression of generation of noise may not be performed due to restriction of cost or the like of a vibration-free member or the like. The second embodiment according to the present invention is also made on assumption that a patient has an uncomfortable feeling due to vibration, and therefore, the second embodiment is preferably combined in one of the methods according to the first embodiment in a case where the uncomfortable feeling of the patient is not reduced.

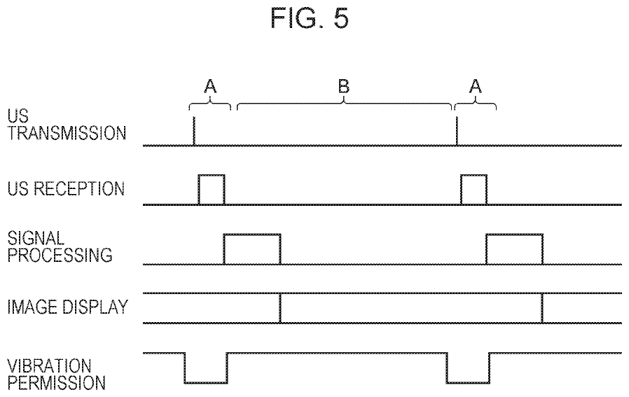

A configuration of a hand-held type ultrasonic probe 1 and an entire block of an imaging apparatus according to the second embodiment are the same as those according to the first embodiment, and therefore, descriptions thereof are omitted. FIG. 5 is a timing chart for facilitating description of the second embodiment of the present invention. In FIG. 5, timings when an ultrasonic image is obtained in an US mode are illustrated. In FIG. 5, an axis of abscissae denotes time. First, a controller 201 of FIG. 4 instructs a timing when the probe element 104 transmits an ultrasonic wave (US transmission). Thereafter, a signal processor 203 converts a signal received by the probe element 104 into a digital signal by an ADC in a period of time in which the ultrasonic wave is transmitted in a distance twice as long as an observation distance (a period of time until the transmitted ultrasonic wave is reflected and returned to the probe element 104) (US reception). Subsequently, the signal processor 203 calculates ultrasonic image data using the obtained digital signal (signal processing). Thereafter, a display unit 204 displays the calculated ultrasonic image data (image display). The processes are performed at substantially the same timings also in a case where a photoacoustic image is obtained in a PA mode. Specifically, the transmission of an ultrasonic wave is replaced by emission of a light pulse. Furthermore, the period of time for the US reception corresponds to a period of time the signal processor 203 converts a signal received by the probe element 104 into a digital signal by the ADC in a period of time in which the ultrasonic wave is transmitted in the observation distance (a period of time until a photoacoustic wave generated due to the light pulse reaches the probe element 104). Subsequently, the signal processor 203 calculates photoacoustic image data using the obtained digital signal (signal processing). Thereafter, a display unit 204 displays the calculated photoacoustic image data (image display).

The controller 201 controls the system by generating such timings for obtaining the various image data. As is apparent from the timing chart of FIG. 5, in a period A (an ultrasonic signal obtaining period), an ultrasonic wave is transmitted or a light pulse is emitted and an ultrasonic wave is received. If vibration is input from an outside in this period, noise is generated. On the other hand, in a period B, the signal processor 203 performs the signal processing (the calculation) and next transmission of an ultrasonic wave or next emission of a light pulse is waited. Even if vibration is input from an outside to the probe element 104 in the period B, a result of the signal processing (the calculation) is not changed. Specifically, even if large vibration is input to the probe element 104, image data to be obtained is not affected. The second embodiment of the present invention makes use of this characteristic, and the microcomputer 110 instructs the vibration actuator 109 to be vibrated only in the period B of FIG. 5. It is more preferable that the microcomputer 110 does not vibrate the vibration actuator 109 until the last moment of the period B but stops vibration of the vibration actuator 109 beforehand taking a period of time required for convergence of vibration of the housing 100 and other structures into consideration.

The microcomputer 110 actually outputs a signal for driving the vibration actuator 109 after performing logical AND on the signal using a vibration permission signal corresponding to the period B (vibration permission in FIG. 5). Furthermore, interruption may be performed in rising of the vibration permission signal so that the vibration actuator 109 is driven in a range in which the period B is not exceeded by the interruption process. According to the second embodiment, generation of noise in an output of the probe element 104 may be suppressed in a period in which an ultrasonic signal is input from the probe element 104 even in a case where generation of noise in an output of the probe element 104 may not be suppressed by the method of the first embodiment or a case where the method of the first embodiment is not performed due to restriction of cost or the like of a vibration-free member 111 or the like, and accordingly, deterioration of image quality of resultant ultrasonic image data or resultant photoacoustic image data may be suppressed.

### Third Embodiment

In the foregoing embodiments, an instruction issued by an operator is input to the hand-held type ultrasonic probe 1 using the pressure sensor 108 corresponding to a switch. In a third embodiment, a pressure sensor 108 may not be required.

According to the third embodiment, as described above, a plurality of connectors are implemented in an imaging apparatus body 2 so that implement of a plurality of hand-held type ultrasonic probes 1 is realized and one of the plurality of hand-held type ultrasonic probes 1 is selected where appropriate in a system. In such an apparatus, a largest problem arises when an operator selects a wrong one of the hand-held type ultrasonic probes 1. If a hand-held type ultrasonic probe 1 is mistakenly selected, an affected area may not be observed, and in addition, the following problem may arise when the hand-held type ultrasonic probe 1 is used in a PA mode. Specifically, if the operator selects a wrong hand-held type ultrasonic probe, a light pulse is emitted from a probe 1 which is to be selected and which is not in contact with the affected area. In this case, the emitted light pulse may be incident on an eye of the operator or the patient.

To address this mistake in which the operator selects a wrong hand-held type ultrasonic probe, according to the third embodiment, if the operator holds a probe 1 other than a hand-held type ultrasonic probe 1 specified by a user interface 200 of an imaging apparatus body 2, a vibration actuator 109 is considerably vibrated so that the operator realizes that the wrong probe 1 is held. If a probe 1 which is not selected is detached from a probe holder, not illustrated, of the imaging apparatus body 2, the imaging apparatus body 2 controls a microcomputer 110 so that the vibration actuator 109 continuously generates large vibration. A determination as to whether a probe 1 has been detached from the probe holder may be easily made by implementing a switch or the like in the probe holder.

Furthermore, if the hand-held type ultrasonic probes 1 have respective connectors, a connector of a probe 1 which is not specified may be disconnected. In this case, information indicating that an inappropriate probe 1 has been selected may not be alerted for the operator by continuous large vibration. To address this problem, if a specified probe 1 is detached from a probe holder of the imaging apparatus body 2, the imaging apparatus body 2 controls the microcomputer 110 of the specified probe 1 so that the vibration actuator 109 is vibrated in a specific vibration pattern. The operator realizes that a wrong probe 1 has not been selected since the operator has knowledge of the specific vibration pattern by well reading an instruction manual. On the other hand, if the probe 1 is not vibrated in the specific vibration pattern, the operator realizes that a wrong probe 1 is held by hand.

According to the third embodiment of the present invention, the operator may determine whether a probe 1 is the specified hand-held type ultrasonic probe 1 only by holding the hand-held type ultrasonic probe 1 by hand. The determination of the hand-held type ultrasonic probe 1 may be made even when the operator is carefully viewing a display unit 204 of the imaging apparatus body 2 or the user interface 200 (without viewing the probe), and therefore, such hand-held type ultrasonic probes 1 are more preferable for the operator. According to the third embodiment of the present invention, in an imaging system including a plurality of hand-held type ultrasonic probes 1, since the vibration actuator 109 is vibrated in a specific pattern indicating a specified probe, the operator may determine whether a probe 1 being held by the operator is the specified probe 1 without interrupting other operations.

### Fourth Embodiment

According to the foregoing embodiments, various information may be transmitted to the operator by implementing the vibration actuator 109 in the hand-held type ultrasonic probe 1 and vibrating the vibration actuator 109 where appropriate. However, while the operator does not hold a probe, such a function may not be useful. Furthermore, if an undesired object is accidentally in contact with the hand-held type ultrasonic probe 1, it is expected that comparatively large sound is output since vibration is enhanced and some may think such sound is uncomfortable. Therefore, a function of stopping vibration of a vibration actuator 109 while the operator does not hold a probe is preferably added. For example, a method for determining that the operator is holding a probe 1 when the probe 1 is not installed in a probe holder which has a switch may be employed. Furthermore, a method for determining whether the probe 1 is being held by the operator in accordance with an output from an infrared sensor or an electrostatic capacitance sensor which is implemented in the hand-held type ultrasonic probe 1 may be employed. By providing such a switch or such a sensor, vibration of the vibration actuator 109 may be suppressed while the operator does not hold the hand-held type ultrasonic probe 1.

Furthermore, in a case where a mode is informed for the operator by vibrating the vibration actuator 109 due to a mode change or the like, if an imaging apparatus has a large number of modes, the vibration actuator 109 may be vibrated first, and immediately after the vibration, a mode name may be displayed in a specific position in a display unit 204. In this case, the vibration actuator 109 notifies the user of information indicating that the vibration indicates display of a mode in the specific position in the display unit 204. If only the display of the mode in the specific position in the display unit 204 is performed, the operator may miss the display since the operator focuses on an obtained image of a test object, for example. However, since the vibration actuator 109 implemented in the hand-held type ultrasonic probe 1 is vibrated, such an oversight may be reduced. At least a notification of the change of a mode for the operator is realized by the vibration of the vibration actuator 109. To realize the notification, a mode name may be displayed in the specific position after the vibration as described above, or normal display and a negative/positive inversion image may be alternately displayed for a while after the vibration, for example. Specifically, a portion for displaying the mode name is at least changed after the vibration.

Although the case where a mode name is displayed is illustrated in the foregoing embodiment, other information to be transmitted by the imaging system to the operator may also be displayed by the same operation.

### Fifth Embodiment

Furthermore, the present invention is applicable to an apparatus which guides a movement of a hand-held type ultrasonic probe disclosed in PTL 2. PTL 2 discloses an apparatus which displays a movement direction of a hand-held type ultrasonic probe in a display device so that a movement operation of the probe is navigated. When the method for performing guiding by image display in a display device is employed, a probe is required to be moved while an operator understands an actual direction of the probe and a direction and a movement amount of the probe displayed in the display device, and accordingly, the operator requires high skill. An audio guide may give an uncomfortable feeling to a patient in a silent environment or may not be used in a noisy environment. Furthermore, a method for attaching a display device constituted by liquid crystals or light emitting diodes to the ultrasonic probe is hardly said to be an excellent guiding method since it is difficult for the operator to view the ultrasonic probe while focusing on an ultrasonic image for diagnosis.

Accordingly, in this embodiment, a mode in which a plurality of vibration actuators 109 are implemented on a grip portion 101 of the hand-held type ultrasonic probe 1 and a movement operation is guided by vibrating one of the vibration actuators 109 corresponding to a movement direction will be described.

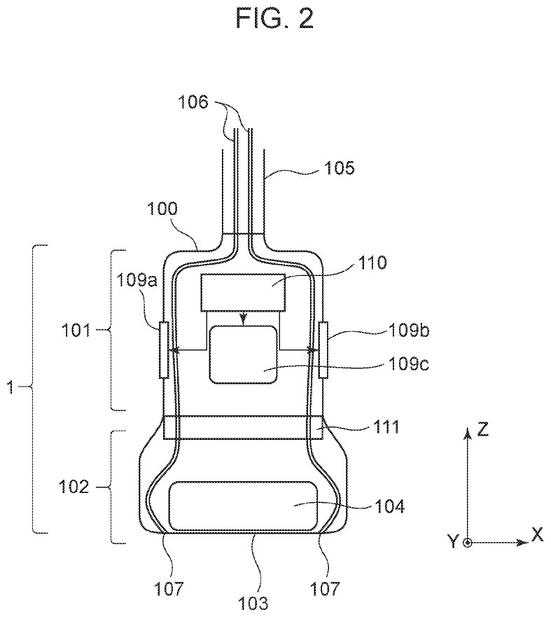

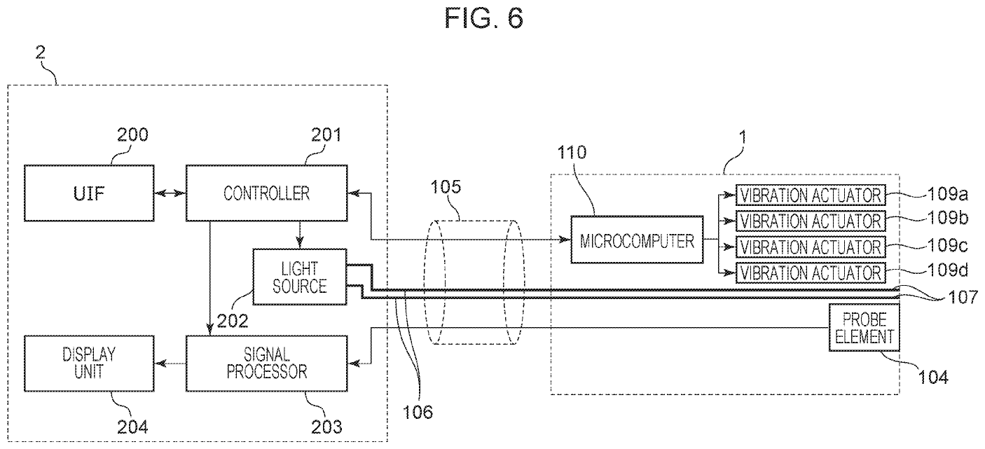

As the hand-held type ultrasonic probe 1 according to this embodiment, a probe 1 illustrated in FIG. 2 is employed. Although the ultrasonic probe 1 illustrated in FIG. 2 is similar to that described in the first embodiment with reference to FIG. 1 except for the plurality of vibration actuators 109. Here, the difference will be mainly described. In FIG. 2, vibration actuators 109a to 109c are illustrated. The vibration actuators 109a and 109b are disposed in an X direction of a grip portion 101 and the vibration actuator 109c and a vibration actuator 109d, not illustrated, are disposed in a Y direction such that the operator feels vibration when holding the grip portion 101 of the hand-held type ultrasonic probe 1. The vibration actuator 109d is implemented on a surface opposite to a surface on which the vibration actuator 109c is disposed in the grip portion 101 of a housing 100. Operations of the vibration actuators 109a to 109d are controlled by a microcomputer 110 in accordance with information supplied from the imaging apparatus body 2 through a cable 105. FIG. 6 is a block diagram illustrating a photoacoustic image obtaining apparatus (an imaging apparatus) using an ultrasonic probe 1 illustrated in FIG. 2. The block diagram of FIG. 6 is similar to that described above according to FIG. 4 except that the four vibration actuators 109a to 109d are illustrated in the hand-held type ultrasonic probe 1.

The imaging apparatus of this embodiment illustrated in FIG. 6 calculates a movement direction and a movement amount of the hand-held type ultrasonic probe 1 which are operation assist information. In this embodiment, instead of the guide using the display unit 204 or the audio guide for transmitting a calculated movement direction and a calculated movement amount of a probe, a movement direction is transmitted to the operator by vibration of the vibration actuators. In this embodiment, a movement direction is specified on an XY plane by vibrating the vibration actuators 109a to 109d. For example, vibration actuators corresponding to an X direction or a Y direction are vibrated when the probe 1 is to be moved to the X direction or the Y direction. Furthermore, when the probe 1 is to be moved to a direction between the X and Y directions, vibration actuators of the corresponding directions are simultaneously vibrated. For example, when a movement direction is to be specified in detail, a magnitude of vibration and a pattern of the vibration are controlled so that a direction of the movement to be performed by the operator is specified. For example, if the vibration actuator 109b in the X direction and the vibration actuator 109c in the Y direction are alternately vibrated in the same ON/OFF time, an angle of 45 degrees which is in the middle of X and Y axes is indicated, and if the vibration actuator 109b and the vibration actuator 109c are vibrated with a time ratio of 1:2, a direction of an angle of 30 degrees relative to a direction of a longer time may be specified. Then, the hand-held type ultrasonic probe 1 is moved in a direction obtained by combining the vibration actuators 109b and 109c. In this way, the direction in the middle of the X and Y axes may be indicated in an analog manner by the vibration of the vibration actuators. Although a direction between the X and Y axes is set in accordance with a period of time the vibration is performed (a vibration duty), the direction between the X and Y axes may be set in accordance with a magnitude of the vibration.

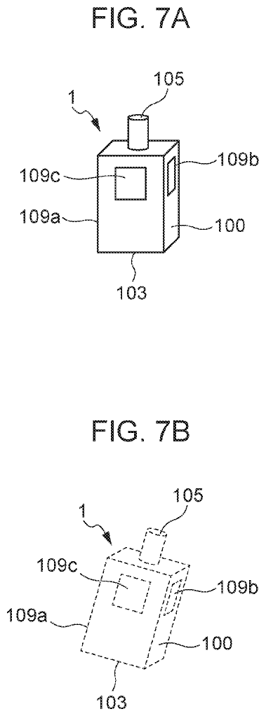

On the other hand, a guide method employed when a probe is moved in an inclined manner will be described below. For simplicity of description, rotation relative to the Y axis (a movement for inclining a positive direction in the X direction toward a -Z direction) is taken as an example for the description. FIGS. 7A and 7B are diagram schematically illustrating a case where the probe 1 is moved in an inclined manner. Reference numerals in FIG. 7 have been described above, and therefore, descriptions thereof are omitted. FIG. 7A is a diagram schematically illustrating a current position of the hand-held type ultrasonic probe 1. FIG. 7B is a diagram illustrating a position to be moved denoted by a dotted line. In this case, when the method for specifying a parallel movement described above is employed, an instruction for movement from a position of the hand-held type ultrasonic probe 1 of FIG. 7A to a position of FIG. 7B may not be issued. In this case, a case where the probe 1 is to be inclined to a front side relative to a surface in which a vibration actuator 109 is implemented is indicated by an operation of repeatedly performing short vibration and short stop, and a case where the probe 1 is to be inclined to a back side is indicated by an operation of repeatedly performing long vibration and short stop. A parallel direction is indicated by an operation of repeatedly performing middle vibration and short stop in a case of a direction for movement. Specifically, both of a parallel movement and an inclination may be instructed by a time length of vibration. For example, an instruction for inclining the hand-held type ultrasonic probe 1 rightward illustrated in FIGS. 7A and 7B may be realized by performing an operation of long vibration and a short interval on the vibration actuator 109a and performing an operation of short vibration and a short interval on the vibration actuator 109b. To set a direction between the X and Y axes as described above, when this inclination instruction is issued, the direction between the X and Y axes may be set by a magnitude of vibration. As described above, a movement of the probe 1 may be instructed (guided) for the operator by a vibration pattern of the vibration actuators. The vibration patterns of the vibration actuators described above are merely examples, and another pattern which is uniquely determined for an instruction may be employed. According to this embodiment, information (a direction for movement of a probe) supplied from an imaging apparatus may be transmitted to the operator by vibration of the vibration actuators implemented on the hand-held type ultrasonic probe 1. According to the present invention, it is effective that a direction for a movement of the hand-held type ultrasonic probe 1 may be more clearly instructed for the operator while the operator focuses on an obtained image when compared with general methods.

### Sixth Embodiment

This embodiment is suitable for a case where an imaging apparatus is to transmit a state of the apparatus or the like (a message) to an operator. Examples of the apparatus state in this embodiment include following states. For example, the examples of the apparatus state include a temperature of a housing, a state of a power-supply voltage, a failure state of a laser device, and the like. As characteristics of the information, information may not be obtained immediately when the operator desires to check the state but the imaging apparatus performs monitoring all the time and notifies the operator of a message only when failure occurs.

This embodiment is realized by the image obtaining apparatus illustrated in FIG. 6 using the probe 1 illustrated in FIG. 2 described in the fifth embodiment. Since the movement direction and the movement amount of the hand-held type ultrasonic probe 1 are not required to be specified according to the second embodiment, only one of the four vibration actuators 109a to 109d illustrated in FIG. 2 may be implemented. When functions of this embodiment are added to the apparatus according to the fifth embodiment, the vibration actuators 109a to 109d are not changed.

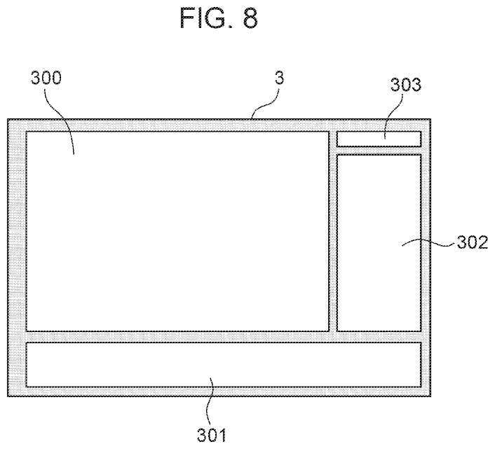

FIG. 8 is a diagram illustrating an example of a display method of a display unit according to this embodiment. In FIG. 8, a display screen 3 is included in the display unit 204, an image display area 300 displays ultrasonic image data and photoacoustic image data, a command area 301 is used to specify an operation of the imaging apparatus by the operator. The operator may issue an operation instruction to the imaging apparatus using a mouse, a graphic user interface (GUI), and a keyboard of the user interface 200 while viewing the command area 301. A status area 302 displays an imaging condition, a histogram, and an analysis result of an obtained information. A message area 303 displays content of a message to be displayed for the operator by the imaging apparatus described above. The message of the imaging apparatus may be transmitted by displaying the message in the message area 303. However, only the display in the message area 303 is performed, the operator may miss the display in the message area 303 when focusing on an image of ultrasonic image data or an image of photoacoustic image data displayed in the image display area 300.

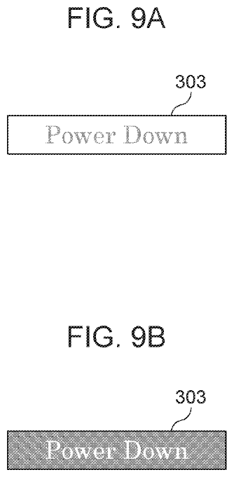

In the method of this embodiment, when the imaging apparatus displays a message in the message area 303, the vibration actuator implemented on the hand-held type ultrasonic probe is activated, and thereafter, a display state of the message in the message area 303 is changed so that the message is transmitted to the operator. Here, the change of the display state of the message is performed within at least several seconds (within 10 seconds at most) immediately after the vibration actuator is vibrated. The display state may be simply changed from a state in which characters to be displayed in the message area 303 are not displayed to a state in which the characters are displayed. Furthermore, as illustrated in FIGS. 9A and 9B, the display state may be changed from a negative display state to a positive display state (negative/positive inversion display) at an interval of 0.5 seconds (FIG. 9B) relative the normal display illustrated in FIG. 9A. Furthermore, in addition to the negative/positive inversion, a change of a display state by a color phase, contrast, or brightness or a change of a display state by a geometric change, such as scrolling or variety of the magnitude, may be employed.

With this configuration, even if the operator is performing diagnosis and focuses on the image display area 300, caution may be issued so that the operator views the predetermined message area 303. As a result, oversight of a message which is transmitted by the imaging apparatus and which is to be viewed by the operator may be avoided. According to the present invention, a display state is changed for several seconds after the vibration actuator is vibrated, and therefore, the operator may realize the message from the imaging apparatus.

As described above, the operator may receive a message without missing the message by the method for vibrating the vibration actuator implemented in the hand-held type ultrasonic probe, and thereafter, transmitting the message to the operator after changing a display state of the message displayed in the message area 303 which is performed by the imaging apparatus. Instead of the method for vibrating the vibration actuator implemented in the hand-held type ultrasonic probe, a method for alerting the operator by beep sound may be employed. However, when the method of the present invention is employed, the operator may find the message from the imaging apparatus even in a location where external noise is large. Furthermore, when the method for alerting the operator by beep sound is employed, the patient may fear the beep sound. However, the present invention may resolve such fear.

### Seventh Embodiment

In this embodiment, a method for implementing a sensor which detects a contact state of a hand-held type ultrasonic probe in the probe and controlling vibration of a vibration actuator by the contact state so that the excellent contact state is maintained will be described.

As disclosed in PTL 3, in a photoacoustic imaging apparatus, a contact surface 103 is required to be in contact with a test object so that a light pulse emitted from a light irradiation unit 107 of a hand-held type ultrasonic probe 1 is not leaked to an outside. In PTL 3, a determination as to whether measurement is to be performed is made after it is determined whether an irradiation light which is leaked from a gap is a safe level for human bodies even if the probe 1 and the test body are not totally in contact with each other. However, it is difficult to easily notify the operator of information indicating one of the probe and the test object which is separated, information indicating a state of the separation, and information indicating one of the probe and the test object to be pressed. For example, even if a portion in the probe to be pressed is displayed in a display unit 204, it is not easy for the operator to perform an operation of improving the contact state based on the display in the display unit 204 depending on a current position and a direction of the probe.

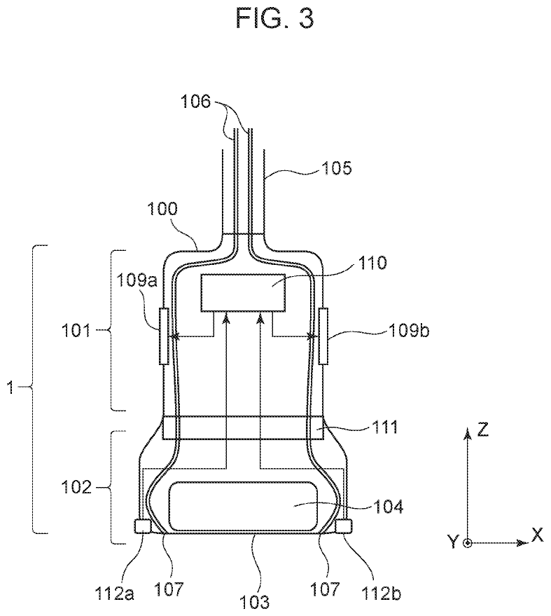

A configuration of the hand-held type ultrasonic probe 1 according to this embodiment is illustrated in FIG. 3. In FIG. 3, descriptions of portions which have reference numerals the same as those described above are omitted. In FIG. 3, reference numerals 112a and 112b denote contact sensors. The contact sensors may be optical sensors disclosed in PTL 3. Any sensor, such as an electrostatic capacitance sensor, may be employed as long as a contact state of a contact surface 103 may be measured. Vibration actuators 109a and 109b are disposed in positions near a cable 105 in a Z direction of the contact sensors 112a and 112b. The two contact sensors 112a and 112b are disposed in an X direction and the respective actuators 109a and 109b are disposed for simplicity of description according to this embodiment. It is preferable that the vibration actuators corresponding to the contact sensors are also implemented in a Y direction. In the configuration of FIG. 3, when the contact sensor 112a detects the separation of the contact surface 103, the corresponding vibration actuator 109a is vibrated. As a result, the operator realizes that a side including the vibration actuator 109a is required to be pressed to the test object, and presses the hand-held type ultrasonic probe 1 as instructed. When the contact sensor detects that the normal contact state of the contact surface 103, the vibration of the vibration actuator 109a is stopped. Similarly, also when the contact sensor 112b is separated from the contact surface 103, the operator realizes the separation by vibration of the vibration actuator 109b and may press a side including the vibration actuator 109b to the test object. In this way, the normal contact state may be easily maintained by pressing the side corresponding to the vibration so that the vibration of the vibration actuators 109a and 109b is stopped. When this method is employed, the operator may intuitively improve the contact state irrespective of a current position and a direction of the probe. Since the normal contact state is maintained, an excellent photoacoustic image may be obtained.

In the foregoing description, the case where the contact surface 103 of the hand-held type ultrasonic probe 1 is separated is assumed. If a problem arises also when the press is too strong, contact sensors 112a and 112b capable of detecting both of states “excessive press” and “separation” are employed. For example, the vibration actuators 109a and 109b are controlled by a microcomputer 110 or a controller 201 such that, when the contact surface 103 is separated, vibration is performed such that long vibration and short stop are repeatedly performed, when the contact surface 103 is excessively pressed, vibration is performed such that short vibration and long stop are repeatedly performed, and when the contact surface 103 is in an appropriate position, vibration is not performed. Since the vibration actuators 109a and 109b are vibrated under this control, the operator may easily press the probe to the test object so that the normal state is attained. Furthermore, for simplicity of description, the configuration in which the contact sensors and the vibration actuators are disposed only in the X direction is described. As described above, contact states in the X and Y direction may be improved by disposing the contact sensors and the vibration actuators also in the Y direction. As described above, according to this embodiment, the user may easily perform an operation of improving a pressing state of the hand-held type ultrasonic probe 1 relative to the separated contact surface 103.

### Eighth Embodiment

In the foregoing embodiment, the vibration actuators are implemented in the hand-held type ultrasonic probe and vibrated where appropriate so that various information is transmitted to the operator. However, if an operator does not hold a probe, such a function may not be useful. Furthermore, it may be assumed that, when another object is in contact with a hand-held type ultrasonic probe, large sound is generated since vibration is increased, and accordingly, the operator may feel uncomfortable. Therefore, a function of stopping vibration of the vibration actuator 109 while the operator does not hold a probe is preferably added. For example, a method for determining that the operator is holding a hand-held type ultrasonic probe 1 when the probe 1 is not installed in a probe holder which has a switch may be employed. Furthermore, a method for determining whether a probe is being held by the operator in accordance with an output from a temperature sensor, an infrared sensor, or an electrostatic capacitance sensor which is implemented in the hand-held type ultrasonic probe may be employed. By providing such a switch or such a sensor, vibration of the vibration actuator 109 may be suppressed while the operator does not hold the probe 1. Furthermore, in this case, various information may not be transmitted to the operator. Therefore, when the operator does not hold the hand-held type ultrasonic probe 1, a large icon, large characters, or the like may be displayed in the display unit 204 in an overlapping manner instead of the vibration of the vibration actuator 109 so that information to be informed is displayed for the operator. Similarly, information to be transmitted to the operator may be indicated by beep sound or the like instead of the vibration of the vibration actuator 109. In this case, the operator has not been made diagnosis on a patient, and therefore, the patient may not be feel fear. Specifically, since this embodiment includes a step of detecting a hand-held type ultrasonic probe held by an operator and if the operator does not hold a probe, a step of vibrating the vibration actuator 109 is omitted. Accordingly, generation of large sound or the like generated due to enlargement of vibration may be suppressed.

First Modification

A configuration described below may be employed as a first modification. According to the foregoing embodiments, the light source 202 is implemented in the imaging apparatus body 2, light is guided to the hand-held type ultrasonic probe 1 by the optical fibers 106, and the light irradiation unit 107 irradiates a test object with the light. When this configuration is employed, the cable 105 becomes large and loses flexibility, and therefore, operability of the hand-held type ultrasonic probe is deteriorated. To address this problem, a light source (an LED array light source) using an LED array or a light source (an LD array light source) using a laser diode array may be implemented in a housing of the probe, and the light irradiation unit 107 may emit a light pulse through an optical member such as an optical fiber. In this case, by implementing an LED array light source or an LD array light source in the reception region portion 102, an optical member and the light irradiation unit 107 may not be implemented.

Second Modification

In the foregoing embodiments, the mode which is realized using the single hand-held type ultrasonic probe 1 or simultaneously using the same hand-held type ultrasonic probes is described. Here, in a case where the embodiments described above are to be simultaneously realized, different vibration patterns may be preferably employed for different functions. It is assumed here that, the vibration of the fifth embodiment is small which is smooth and the vibration of the sixth embodiment is comparatively large and lasts for a short period of time so that a clicking feeling is obtained. It is assumed further that the vibration of the seventh embodiment has a magnitude between the vibration of the fifth embodiment and the vibration of the sixth embodiment. The vibration patterns are merely examples, and any vibration pattern may be employed as long as the operator easily distinguishes the vibration pattern.

The present invention is not limited to the foregoing embodiment, and various changes and modifications may be made without departing from the spirit and the scope of the present invention. Accordingly, claims below are attached to disclose the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An ultrasonic probe including a housing having a reception region portion and a grip portion,
wherein the reception region portion includes an ultrasonic element array which receives ultrasonic waves and the grip portion includes a vibration actuator.

2. The ultrasonic probe according to claim 1, wherein a vibration-free mechanism which reduces vibration transmitted to the reception region portion from the vibration actuator is disposed between the reception region portion and the grip portion.

3. The ultrasonic probe according to claim 1, wherein the reception region portion includes a light irradiation unit which irradiates a test object with light.

4. The ultrasonic probe according to claim 1, wherein the reception region portion includes a light irradiation unit which irradiates a test object with light, and the ultrasonic element array included in the reception region portion transmits the ultrasonic waves.

5. The ultrasonic probe according to claim 4, wherein different vibration patterns of the vibration actuator are obtained in different modes including a first mode which performs transmission and reception of ultrasonic waves relative to the reception region portion and a second mode in which the reception region portion receives photoacoustic waves generated in the test object due to the light irradiation from the light emission terminal.

6. The ultrasonic probe according to claim 4, wherein, in a case where the first and second modes are simultaneously operated, vibration may be performed in a third vibration pattern which is different from a first vibration pattern in which the vibration actuator is vibrated during the operation in the first mode and a second vibration pattern in which the vibration actuator is vibrated during the operation in the second mode.

7. The ultrasonic probe according to claim 1, wherein the vibration actuator is vibrated in a period of time which is different from a period of time in which the ultrasonic waves are received.

8. The ultrasonic probe according to claim 1, wherein the vibration actuator is vibrated in accordance with a state of contact with the test object or a change of the contact state.

9. The ultrasonic probe according to claim 1, wherein a frequency band used by the ultrasonic element array for reception of ultrasonic waves is different from a frequency band for vibrating the vibration actuator.

10. The ultrasonic probe according to claim 9, wherein, when a maximum sensitivity of the frequency band used for reception of ultrasonic waves by the ultrasonic element array is set to 100, the vibration actuator is vibrated in a frequency band corresponding to 10% of the maximum sensitivity or less.

11. The ultrasonic probe according to claim 10, wherein the vibration actuator is vibrated in a frequency band corresponding to 3% of the sensitivity or less.

12. The ultrasonic probe according to claim 9, wherein a frequency band used by the ultrasonic element array for transmission of ultrasonic waves is different from a frequency band for vibrating the vibration actuator.

13. The ultrasonic probe according to claim 1, wherein an LED array or LD array which emits light from the light irradiation unit are incorporated in the housing.

14. The ultrasonic probe according to claim 13, wherein the LED array or the LD array is disposed on the reception region portion in the housing.

15. The ultrasonic probe according to claim 1, wherein the ultrasonic element array is an array of ultrasonic transducers.

16. The ultrasonic probe according to claim 15, wherein the ultrasonic transducer is electrostatic capacitance transducer.

17. The ultrasonic probe according to claim 15, wherein a piezoelectric element is used for the ultrasonic transducer.

18. An ultrasonic image obtaining apparatus, comprising:
the ultrasonic probe according to claim 1;
a signal processor configured to generate image data using a signal based on a ultrasonic wave received by the ultrasonic probe; and
a display unit configured to display an image based on the image data.

19. The ultrasonic image obtaining apparatus according to claim 18, wherein a body of the ultrasonic image obtaining apparatus includes the signal processor and the display unit and the ultrasonic probe is connected to the body through a cable.

20. The ultrasonic image obtaining apparatus according to claim 19, wherein the cable has a connector and the ultrasonic probe connected to the cable through the connector is connected to the apparatus body.

21. The ultrasonic image obtaining apparatus according to claim 20, wherein different ultrasonic probes having different characteristics are individually connected to the cable, and the ultrasonic probes are connected to the apparatus body with the cable in a replacement available manner.
